(19) United States
(12) Patent Application Publication
(10) Pub. No.: US 2014/0275844 A1
(43) Pub. Date: Sep. 18, 2014

(54) Universal Patient Interface Module and Associated Devices, Systems, and Methods

(71) Applicant: Volcano Corporation, San Diego, CA (US)

(72) Inventors: Paul Michael Hoseit, El Dorado Hills, CA (US); Paul Douglas Corl, Palo Alto, CA (US); Sherwood Kantor, Sacramento, CA (US)

(73) Assignee: Volcano Corporation, San Diego, CA (US)

(21) Appl. No.: 14/212,894

(22) Filed: Mar. 14, 2014

Related U.S. Application Data
(60) Provisional application No. 61/800,489, filed on Mar. 15, 2013.

(51) Int. Cl. A61B 5/00; A61B 5/027; A61B 1/00; A61B 8/00; A61B 8/08; A61B 5/0205; A61B 8/12

(52) U.S. Cl. CPC A61B 5/0084; A61B 5/0205; A61B 5/027; A61B 5/6852; A61B 8/12; A61B 8/4461; A61B 5/0066; A61B 8/52; A61B 5/725; A61B 1/00165
USPC 600/301; 600/407

(57) ABSTRACT

An intravascular device interface and associated systems and methods are disclosed. In some embodiments, the intravascular device interface includes a housing containing one or more processors in communication with a memory, a first connector, and a second connector. The first connector is secured to the housing and configured to interface with a proximal connector of an imaging intravascular device sized and shaped for insertion into human vasculature. The second connector is also secured to the housing and is configured to interface with a proximal connector of a physiology intravascular device. The one or more processors process data received from the first connector or the second connector for transmission over a device output. In some embodiments disclosed herein, an intravascular device interface includes a single connector that may be configured to support a non-rotational imaging device or a rotational imaging device.

### CROSS-REFERENCE TO RELATED APPLICATIONS

The present application claims priority to and the benefit of U.S. Provisional Patent Application No. 61/800,489, filed Mar. 15, 2013, which is hereby incorporated by reference in its entirety.

### TECHNICAL FIELD

The present disclosure is related to device interfaces and associated systems and methods used in conjunction with a plurality of different types of intravascular devices.

### BACKGROUND

Innovations in diagnosing and verifying the level of success of treatment of disease have migrated from external imaging processes to internal diagnostic processes. In particular, diagnostic equipment and processes have been developed for diagnosing vasculature blockages and other vasculature disease by means of ultra-miniature sensors placed upon the distal end of a flexible elongate member such as a catheter or a guide wire used for catheterization procedures. For example, known medical sensing techniques include angiography, intravascular ultrasound (IVUS), forward looking IVUS (FL-IVUS), pressure measurement for fractional flow reserve (FFR) determination, flow measurement for coronary flow reserve (CFR) determination, optical coherence tomography (OCT), trans-esophageal echocardiography, and image-guided therapy. Each of these techniques may be better suited for different diagnostic situations. To increase the chance of successful treatment, health care facilities may have a multitude of imaging and sensing modalities on hand in a catheterization laboratory (cath lab) during a procedure. However, each imaging modality in a cath lab traditionally requires its own special-purpose diagnostic equipment. For instance, each imaging modality may require a catheter, a patient interface module (PIM), a user control interface, a display, a specialized power unit, and a processing unit such as a customized personal computer. Traditionally, all of this equipment is located in the cath lab itself during a procedure and depends on a substantial wiring infrastructure for network connectivity and dependable power. Physical space is typically at a premium in cath labs and each additional imaging modality employed in a cath lab complicates the pre-procedure setup and limits the movement of health care professionals during procedures. Additionally, known problems arise when attempting to locate portions of a diagnostic system outside of the cath lab. For instance, analog data transmitted between a bedside PIM and a processing unit may degrade as the distance between the two increases. Similarly, traditional long-distance communication links do not support the bandwidth required for modern cardiovascular imaging techniques.

While the existing devices and methods have been generally adequate for their intended purposes, they have not been entirely satisfactory in all respects. The medical sensing systems and associated methods of the present disclosure overcome one or more of the shortcomings of the prior art.

### SUMMARY

Patient interface modules and associated systems and methods are provided that facilitate the use of a plurality of different intravascular devices using a single patient interface module, instead of having separate patient interface modules for each type of intravascular device.

In some embodiments, an intravascular device interface is provided. The intravascular device interface includes a housing containing one or more processors in communication with a memory, and first and second connectors secured to the housing. The first connector is configured to interface with a proximal connector of an imaging intravascular device, which is sized and shaped for insertion into human vasculature. The second connector is configured to interface with a proximal connector of a physiology intravascular device, which is also sized and shape for insertion into human vasculature. The one or more processors process data received from the first connector or the second connector for transmission over a device output.

In some embodiments, another intravascular device interface is provided. The device interface or interface device includes a controller having one or more processors in communication with a memory and a first connector configured to communicatively receive an intravascular imaging device. When the intravascular imaging device is a rotational intravascular imaging device and is coupled to the first connector, the rotational intravascular imaging device is coupled to a motor enabled to rotate at least a portion of the rotational intravascular imaging device. When the intravascular imaging device is a non-rotational intravascular imaging device and is coupled to the first connector, a rotation prevention mechanism is activated to prevent rotation of the non-rotational intravascular imaging device.

In some embodiments, an intravascular device system is provided. The intravascular device system includes an intravascular device interface that has a first connector configured to interface with a proximal connector of an imaging intravascular device and a second connector configured to interface with a proximal connector of a physiology intravascular device. The intravascular device system further includes a rotational handpiece coupled to the intravascular device interface, a sled supporting the intravascular device interface so that the intravascular device interface is controllably moveable along an axis, and a plurality of user controls in communication with the rotational handpiece.

In some embodiments, another intravascular device system is provided. The intravascular device system includes an intravascular imaging device that has a flexible elongate member sized and shaped more insertion into human vasculature and an intravascular device interface. There intravascular device interface is coupled to the intravascular imaging device, and has a first connector by which the intravascular device interface is coupled to the intravascular imaging device. The intravascular device interface is configured such that when the intravascular imaging device is a rotational intravascular imaging device, the intravascular imaging device is coupled to a motor enabled to rotate at least of portion of the rotational intravascular imaging device, and when the intravascular imaging device is a non-rotational intravascular imaging device, a rotation prevention mechanism is activated to prevent rotation of the non-rotational intravascular imaging device. The intravascular device system further includes a sled supporting the intravascular device interface so that the intravascular device interface can be controllably moved along an axis.

In some embodiments, a method for using an intravascular imaging device to gather imaging data from within a patient is provided. The intravascular imaging device being coupled to an intravascular device interface, and the method includes steps of receiving an intravascular imaging device in a first connector provided on the intravascular device interface, of determining whether the intravascular imaging device received in the first connector is a rotational intravascular imaging device or a non-rotational intravascular imaging device, and of configuring operational features on the intravascular device interface depending on the determination of whether the intravascular imaging device is a rotational intravascular imaging device or a non-rotational intravascular imaging device.

In some embodiments, a method for collecting multiple types of data from multiple intravascular device types within a patient is provided. The method includes steps of coupling an intravascular imaging device to an intravascular device interface and of coupling an intravascular physiology-sensing device to the intravascular device interface. The intravascular imaging device and the intravascular physiology-sensing device are coupled to the intravascular device interface during a data collection period. The method further includes steps of collecting imaging data using the intravascular imaging device, of collecting physiological data using the intravascular physiology-sensing device, and of processing the imaging data and the physiological data using one or more processors provided by the intravascular device interface.

These and other aspects of the present disclosure will be described in further detail below with respect to the following figures.

### BRIEF DESCRIPTION OF THE DRAWINGS

FIG. 1 is a diagram of a medical system for treating a patient according to an embodiment of the present disclosure.

FIG. 2 depicts an intravascular device system according to an embodiment of the present disclosure.

FIG. 3 depicts an intravascular device interface with multiple intravascular device connectors according to an embodiment of the present disclosure.

FIG. 4 depicts an intravascular device system with multiple intravascular device connectors according to another embodiment of the present disclosure.

FIG. 5 depicts a schematic diagram of a sled for an intravascular device interface of the intravascular device system of FIG. 4 according to an embodiment of the present disclosure.

FIG. 6 depicts a schematic diagram of a user controls component of the intravascular device system of FIG. 4 according to an embodiment of the present disclosure.

FIG. 7 depicts a schematic diagram of a rotational handpiece of the intravascular device system of FIG. 4 according to an embodiment of the present disclosure.

FIG. 8 depicts a schematic diagram of a bedside interface box of the intravascular device system of FIG. 4 according to an embodiment of the present disclosure.

FIG. 9 depicts an intravascular device system according to an additional embodiment of the present disclosure.

FIG. 10 depicts an intravascular device interface according to an additional embodiment of the present disclosure.

FIG. 11 is a flowchart of a method for using an intravascular imaging device to gather imaging data from within a patient according to an embodiment of the present disclosure.

FIG. 12 is a flowchart of a method for collecting multiple types of data from multiple intravascular device types according to an embodiment of the present disclosure.

For clarity of discussion, elements having the same designation in the drawings may have the same or similar functions. The drawings may be better understood by referring to the following Detailed Description.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

“Connected”, “coupled”, and variations thereof as used herein includes direct connections, such as being glued or otherwise fastened directly to, on, within, etc. another element, as well as indirect connections where one or more elements are disposed between the connected elements.

“Intravascular device”, as used herein includes hypo tubes, catheters, guide wires, and other devices such as may be used by medical personnel to provide various treatments, and to obtain data from vessels throughout the body.

FIG. 1 is a schematic diagram depicting a medical system 100 including a medical data processing system 101 according to an embodiment of the present disclosure. In general, the medical system 100 provides for coherent integration and consolidation of multiple forms of acquisition and processing elements designed to be sensitive to a variety of methods used to acquire and interpret human biological physiology and morphological information and to coordinate treatment of various conditions. As depicted in FIG. 1, the medical system 100 is partially situated in a cath lab room 102 and partially in an associated control room 104.

In one embodiment, the processing system 101 is a console with the hardware and software to acquire, process, and display medical data, but, in other embodiments, the processing system 101 may be any other type of computing system operable to process medical data. In the embodiments in which processing system 101 is a console or computer workstation, the system includes one or more processors such as a microcontroller and/or a dedicated central processing unit (CPU), a non-transitory computer-readable storage medium or memory such as a hard drive, random access memory (RAM), and/or compact disk read only memory (CD-ROM), a video controller such as a graphics processing unit (GPU), and a network communication device such as an Ethernet controller or wireless communication controller. The processing system 101 displays visualized data using its video controller in communication with a display 106.

In some embodiments, the processing system 101 is programmed to execute steps associated with the data acquisition, analysis, and control described herein. Accordingly, it is understood that any steps related to data acquisition, data processing, instrument control, and/or other processing or control aspects of the present disclosure may be implemented by the processing system 101 using corresponding instructions stored on or in a non-transitory computer readable medium, or memory, accessible by the processing system. In some instances, the processing system 101 is portable (e.g., handheld, on a rolling cart, etc.). Further, it is understood that in some instances processing system 101 comprises a plurality of computing devices, such that the steps of data acquisition, analysis, and control described herein may be shared or distributed across the plurality of computing devices. In that regard, it is particularly understood that the different processing and/or control aspects of the present disclosure may be implemented separately or within predefined groupings using a plurality of computing devices. Any divisions and/or combinations of the processing and/or control aspects described below across multiple computing devices are within the scope of the present disclosure.

As depicted in FIG. 1, intravascular devices 108 and 110 are included in the medical system 100. Intravascular devices 108 and 110 are medical sensing devices that are sized and shaped for insertion into human vasculature and may be utilized by a clinician to acquire medical sensing data about the patient 112. In a particular instance, the intravascular device 108 collects medical sensing data using one modality and the intravascular device 110 collects medical sensing data using a different modality. For instance, the intravascular devices 108 and 110 may each collect data related to one or more of pressure, flow (velocity), temperature, images (including images obtained using ultrasound (e.g., intravascular ultrasound (IVUS), and differing modalities thereof, including rotational and solid-state IVUS at various operating frequencies), optical coherence tomography (OCT), thermal, and/or other imaging techniques), and/or combinations thereof. The intravascular devices 108 and 110 may be any form of device, instrument, catheter, or probe that is sized and shaped to be positioned within a vessel, including within human vasculature. In some embodiments, one or both of the intravascular devices 108 and 110 may be a steerable device, either on its own or as inserted into a steerable intravascular device sheath. Additional examples of intravascular imaging devices and/or associated features that may be used in embodiments herein can be found in U.S. Pat. No. 7,967,762, filed Jan. 4, 2007 and titled “ULTRA MINIATURE PRESSURE SENSOR;” U.S. Pat. No. 6,641,540, filed Sep. 6, 2001 and titled “MINIATURE ULTRASOUND TRANSDUCER”; U.S. Pat. No. 6,511,432, filed Apr. 27, 2001 and titled “PREAMPLIFIER AND PROTECTION CIRCUIT FOR AN ULTRASOUND CATHETER;” U.S. Pat. No. 5,546,948, filed Nov. 28, 1994 and titled “ULTRASOUND IMAGING GUIDEWIRE;” U.S. Pat. No. 5,243,988, filed Aug. 7, 1992 and titled “INTRAVASCULAR IMAGING APPARATUS AND METHODS FOR USE AND MANUFACTURE;” U.S. Provisional Patent Application No. 61/695,970, filed Aug. 31, 2012 and titled “MOUNTING STRUCTURES FOR COMPONENTS OF INTRAVASCULAR DEVICES;” and U.S. Provisional Patent Application No. 61/665,697, filed on Jun. 28, 2012 and titled “INTRAVASCULAR DEVICES, SYSTEMS, AND METHODS;” each of which is hereby incorporated by reference in its entirety.

In some embodiments, more than one modality may be implemented in a single intravascular device. For example, intravascular device 108 may be configured to collect multiple modalities of physiology data, such as flow and pressure data. In such an instance, the intravascular device 108 has both a pressure sensor and a flow sensor disposed in such a way to gather data from within patient 112. As another example, the intravascular device 108 is capable of multi-modality image sensing in some instances, such as both IVUS and intravascular photoacoustic (IVPA) sensing, or IVUS and OCT, and/or other combinations. In some embodiments of medical system 100, intravascular device 108 is an intravascular physiology device and the intravascular device 110 is an intravascular imaging device. The intravascular device 100 may be a rotational intravascular imaging device or a solid-state (non-rotational) intravascular imaging device. In yet other embodiments, both intravascular devices 108 and 110 are intravascular imaging devices. In such embodiments, intravascular device 108 may be a rotational IVUS device incorporating a piezoelectric micromachined ultrasound transducer (PMUT) or a traditional piezoceramic ultrasound transducer, and intravascular device 110 may be a non-rotational IVUS device, such as a solid-state IVUS device. In that regard, in some instances one or both of the intravascular devices 108 and 110 are IVUS imaging devices available from Volcano Corporation, such as the Eagle Eye® Platinum, Eagle Eye® Platinum ST, Eagle Eye® Gold, Revolution®, Visions® PV, and/or other IVUS imaging devices.

The intravascular devices 108 and 110 may be used individually or collectively during a given procedure. In some embodiments in which intravascular devices 108 and 110 are both intravascular imaging devices and connect to a common port on an interface device, either intravascular device may be used during a given procedure or both may be used sequentially during the procedure. In embodiments in which intravascular device 108 is an intravascular physiology device and the intravascular device 110 is an intravascular imaging device connected to separate ports on an interface device, both intravascular devices may be used to gather data at the same time during a same procedure.

In the depicted embodiment, an intravascular device interface 114, which also may be referred to herein as a patient interface module (PIM) or a bedside interface box (BIB), connects the intravascular device 108 and 110 to other components within the medical system 100. The intravascular device interface 114 is configured to support (e.g., collect data and status information from and transmit instructions and/or control signals to, both intravascular devices 108 and 110). In some embodiments, intravascular device interface 114 includes a connector that is configured to provide an interface for both intravascular devices 108 and 110 in sequence; while in other embodiments intravascular device interface 114 includes a connector to support intravascular device 108 and a separate connector to simultaneously support intravascular device 110. The intravascular device interface 114 is a universal intravascular device interface in the sense that is supports more than one intravascular device type as will be disclosed in greater detail throughout the disclosure. In FIG. 1, intravascular device interface 114 is depicted as coupled to processing system 101 and to a bedside controller or console 116. In some embodiments, information obtained from one or both of intravascular devices 108 and 110 is transmitted to console 116, which then displays at least some of the information on display 106, which may be a monitor, touchscreen, tablet, heads-up display, television, and/or other component capable of visually displaying information to a user.

The console 116 may be used by an operator to transmit instructions to the intravascular device interface 114 for implementation by one or both of intravascular devices 108 and 110. The console 116 is also communicatively coupled to the processing system 101 and provides user control of the particular medical modality (or modalities) being used to diagnose the patient 112. In some embodiments, the bedside console 116 is a touch screen controller that provides user controls and diagnostic images on a single surface. In alternative embodiments, however, the bedside console 116 may include both a non-interactive display and separate controls such as physical buttons (e.g., keyboard, mouse, etc.) and/or a joystick and may process or further process data received from intravascular devices. In the medical system 100, the bedside console 116 is operable to present workflow control options and patient image data in graphical user interfaces (GUIs).

In some embodiments of medical system 110, a main controller or console 120 is situated in the control room 104 adjacent to the catheter lab 102 and is also communicatively coupled to the processing system 101. In the current embodiment, the main console 120 is similar to the bedside console 116 in that it includes a touch screen and is operable to display multitude of GUI-based workflows corresponding to different medical sensing modalities.

While in the depicted embodiment intravascular device interface 114 is in communication with the processing system 101 as described above, in some embodiments, the bedside controller 116 receives data from and communicates commands to the intravascular device interface 114. In some embodiments, the main controller 120 additionally or alternatively receives data from and communicates commands to the intravascular device interface 114.

With reference now to FIG. 2, shown therein is an intravascular device system 200 according to an embodiment of the present disclosure that shares many of the components and features described above in connection with the medical system 100 of FIG. 1. Intravascular device system 200 may be used within the context of medical system 100. For example, included in the intravascular device system 200 is a display 106 in communication with a console 116 similar to those depicted in FIG. 1. The intravascular device system 200 further includes an intravascular device interface 202 that may be used as the intravascular device interface 114 of FIG. 1. The intravascular device interface 202 is disposed on a sled 204 that allows the intravascular device interface 202 to be controllably moved along an axis determined by the sled 204. By moving the intravascular device interface 202 along an axis determined by sled 204, a distal end of an intravascular device may be controllably moved within a patient such as patient 112 of FIG. 1. However, in some embodiments of intravascular device system 200, sled 204 is absent or is present but does not provide controlled movement along an axis.

As depicted in the embodiment shown in FIG. 2, the intravascular device interface 202 includes a first intravascular device connector 206 in addition to a second intravascular device connector 208. In other embodiments of the intravascular device interface 202, more than two connectors may be present. As depicted, first connector 206 is coupled to an intravascular device 216, while the second connector 208 is coupled to an intravascular device 218. In general, the intravascular devices 216 and 218 are different types or modalities of intravascular devices. As discussed above in connection with intravascular devices 108 and 110 of FIG. 1, in one embodiment of the intravascular device system 200 the intravascular device 216 is an intravascular imaging device, such as an IVUS device or an OCT device. The intravascular device 218 may be an intravascular physiology device configured to collect flow data, pressure data, and/or other physiological data. In such an embodiment, first connector 206 is configured to support an intravascular imaging device while second connector 208 is configured to support an intravascular physiology device.

Some embodiments of the intravascular device interface 202 are configured to process the data obtained from the intravascular imaging device 216 through the first connector 206 and from the intravascular physiology device 218 through the second connector 208. Intravascular device interface 202 includes one or more processors, including a signal processor or signal processing circuit, that can be used to process data for transmission over a link or cable 220 that couples the intravascular device interface 202 to console 116 and/or display 106. In some embodiments, intravascular device interface 202 processes obtained data to such an extent that only display data is transmitted over cable 220 requiring relatively little bandwidth, while in other embodiments data is transmitted over cable 220 for additional processing by console 116. As depicted in FIG. 2, the cable 220 is a fiber optic cable that is used to transmit fiber optic signals between console 116 and the intravascular device interface 202. In some instances, the intravascular device interface 202 communicates wirelessly with the console 116.

Some embodiments of the intravascular device system 200 include a third connector configured to provide an interface for a third intravascular device. In some such embodiments, the first connector is configured to provide an interface for a rotational intravascular imaging device, while the third connector is configured to provide an interface for a non-rotational intravascular imaging device.

Now referring to FIG. 3, shown therein is an intravascular device interface 300 similar to the intravascular device interface 202 of FIG. 2 and the intravascular device interface 114 of FIG. 1. The intravascular device interface 300 is shown as having a plurality of intravascular device connectors. Depicted are an intravascular device connector 302A, an intravascular device connector 302B, and an intravascular device connector 302N, representing that embodiments of the intravascular device interface 300 may have any number of intravascular device connectors. The intravascular device connectors 302A-N are configured in communication with a data processor 304 and a control processor 306. In general the intravascular device interface 300 may include one or more processors, with some processors being allocated specific tasks or with tasks being distributed among the processors. In some embodiments, the one or more processors, including data processor 304 and control processor 306 are implemented as a configuration of a field-programmable gate array (FPGA), a programmable logic device (PLD), or another programmable processor. In some embodiments, the data processor 304 and the control processor 306 are provided by a single processor. In embodiments of the intravascular device interface 300 that include a programmable processor, one or more configurations of the processor may be stored in a memory 308, from which they can be implemented as needed.

The memory 308 may be a hard disk drive, a solid-state drive, RAM, or other type of memory device that can hold instructions and/or data. For example, data may be gathered from an intravascular device connected through the intravascular device connector 302A to the data processor 304 and/or the control processor 306, which may store some or all of that data in memory 308 before, during, and/or after data or signal processing is performed by the data processor 304. After signal processing is performed on the data in memory 308, the control processor 306 may cause the processed data to be transmitted from memory 308 to an external device such as the console 116 of FIG. 2 and FIG. 1 through a data output 310. As has been discussed, the data output 310 in some embodiments is a fiber optic data output including a small form factor pluggable transceiver. However, not all embodiments of the intravascular device interface 300 include a fiber optic data output for the data output 310.

In addition to outputting data through the data output 310, the intravascular device interface 300 is configured to receive control instructions and queries through a control input 312. Depending on the particular embodiment of the intravascular device interface 300, the control input 312 may be configured to interface with a handpiece controller, a controller provided in communication with a console such as a keyboard, a mouse, or a touch screen display, or from another controller. The intravascular device interface 300 further includes a power supply 314 that is configured to supply power internally to the intravascular device interface 300, e.g. to components including the data processor 304, the control processor 306, the memory 308 and other components. Power supply 314 is further configured to provide power through the intravascular device connectors 302A-N to any and all intravascular devices coupled thereto.

Referring now to FIG. 4, shown therein is an intravascular device system 400 that includes a number of components. As shown in FIG. 4, a sled 500 (which is similar to the sled 204 of FIG. 2) is coupled by a link 402 to a rotational handpiece 700. The rotational handpiece 700 supports a set of user controls 600 shown coupled by a link 404. In some embodiments, the user controls 600 may be part of a user interface incorporated into the rotational handpiece 700; while in other embodiments, the user controls 600 are made available through an external console or similar device. The user controls 600 allow a user of the intravascular device system 400 to selectively collect imaging data, gather physiology data, control the sled 600 for pullback movements, and perform other functions.

The rotational handpiece 700 is configured to cooperate with a rotational intravascular device, such as a rotational imaging catheter, and is in communication with a patient interface module or bedside interface box (BIB) 800. Referred to herein as BIB 800, the bedside interface box shares many features with the intravascular device interfaces 202 of FIGS. 2 and 300 of FIG. 3. In the embodiment depicted in FIG. 4, BIB 800 includes a plurality of connectors. These connectors include an intravascular physiology device connector 310, a solid-state imaging device connector 320, and a rotational imaging device handpiece connector 830. As shown in FIG. 4, the rotational handpiece 700 is coupled to the connector 830 providing a communication link 406 permitting communication between the rotational handpiece 700 and the BIB 800.

The intravascular physiology device connector 810 is configured to provide an interface for intravascular devices having a pressure sensor, a flow sensor, or both of a flow sensor and a pressure sensor. The solid-state imaging device connector 820 is configured to interface with solid-state imaging devices, such as a phased array IVUS device like the EAGLE EYE® Platinum catheter produced by Volcano Corporation of San Diego, Calif. The intravascular rotational imaging device connector 830 is an interface for rotational imaging devices such as an OCT device or an IVUS device like the REVOLUTION® 45 MHz catheter also produced by the Volcano Corporation. In some embodiments of the intravascular device system 400, a rotational intravascular imaging device may be coupled directly to the connector 830 of the BIB 800 rather than through the rotational handpiece 700. In such embodiments, the sled 500 may be controlled by the user controls 600 as provided by BIB 800 or by an external console, such as console 116 of FIGS. 1 and 2. More detail regarding embodiments of the main components depicted in the intravascular device system 400 of FIG. 4 can be found in FIGS. 5, 6, 7, and 8 and are discussed in detail below.

Referring now to FIG. 5, shown therein is the sled 500 as also depicted in FIG. 4. As depicted in FIG. 5, the sled 500 is configured to communicate with the rotational handpiece 700 over the communication link 402 that is established through a physical interface 502. The physical interface 502 provides connections to a plurality of control, data, and power lines to the components of sled 500. Sled 500 includes a stepper motor 504 that, in response to instructions received over the communication link 402 through the physical interface 502, controllably moves the BIB 800 either forward or backward a controlled distance and at a controlled rate along an axis provided by sled 500. Sled 500 further includes an encoder 506 configured to communicate position data over the communication link 402.

Referring now to FIG. 6, shown therein is the user controls 600 also depicted in FIG. 4. The user controls 600 are shown in communication over the communication link 404 with the rotational handpiece 700. The user controls 600 includes a physical interface 602 that, in turn, includes a plurality of communication lines to and from components of the user control 600. In the depicted embodiment of the user controls 600, a user control panel 604 is used to communicate with an operator of the intravascular device system 400. As depicted, the user control panel 604 includes a plurality of physical buttons allowing a user to exert control and a plurality of light-emitting diode (LED) indicators that communicate various functions and conditions to the user. For example, the user control panel 604 may receive commands from a user to begin imaging, to save a frame, to cause the sled 500 to execute a pullback sequence, to reset settings, and or to switch between an automatic mode and a manual mode. In some embodiments, the user control panel 604 is a touch-screen display panel that provides a plurality of touchable buttons and LED-like indicators for communication with the operator. The physical interface 602 also provides power and ground to the user control panel 604. As depicted in FIG. 6 and in FIG. 4, the user controls 600 are configured to be in direct communication with the handpiece 700. In some embodiments, the user controls 600 are provided by the BIB 800 or by a console in communication therewith, such as console 116 or 120 of FIG. 1 or FIG. 2.

Referring to FIG. 7, an embodiment of a rotational handpiece 700 is shown therein. As depicted, the rotational handpiece 700 is in communication with the sled 500, the user controls 600, and the BIB 800 over communication links 402, 404, and 406, respectively. Associated with each of these links, there is a physical interface providing physical conductive lines for the exchange of electronic signals and power, and there is also an electrostatic discharge protection system. Power is received over the communication link 406 from the BIB 800 and is converted and filtered by power circuit 702 to the levels required by the controller 704, spinner motor 706, and to a number of other components depicted in FIG. 7. Power circuit 702 also distributes power to the sled 500 and the user controls 600. Additionally, power circuit 702 provides fault monitoring and shut down capability to disable power in the event of component or subassembly failure. Power circuit 702 can be configured to receive a wide range of DC input voltages from for example 5 Volts to 30 Volts which it converts to the required DC supply voltages such as +12 Volts, +5 Volts, −5 Volts, +3.3 Volts, +2.5 Volts, +1.8 Volts, +1.2 Volts, +1.0 Volts, and +0.75 Volts. The power circuit 702 also provides a programmable high voltage supply (Transmit HV) which provides a range of DC voltages from 40 Volts to 90 Volts for use by the ultrasound pulser circuit that may reside within the IVUS catheter or within the handpiece.

As depicted, the controller 704 is implemented as one or more processors configured to process and respond to control information and requests and to process data for use internally and externally by the rotational handpiece 700. In some embodiments, the controller 704 is provided by a field-programmable gate array (FPGA) that can be configured with a particular configuration in response to a type of intravascular device connected to the device connector 708, to the slip rings 710, and/or to the spinner motor 706. Controller 704 is made aware of a functional coupling to intravascular device by the triggering of an intravascular device detection switch 712 coupled thereto. Upon receiving a device detect switch signal, the controller 704 may read information associated with the inserted intravascular device (e.g., from an EEPROM, RFID tag, bar code, and/or other electronic or physical representation of information) in order to determine a type of the device. The information may contain a device identification value identifying the type of the device and/or associated operating or calibration parameters. Other identifying characteristics may also be used to determine the type of the intravascular device. When in operation, a position of the coupled intravascular device may be determined from information received from encoder 714 coupled to the spinner motor 706 and to the controller 704. If an adjustment in the rotational position of the coupled intravascular device is to be made, the controller 704 sends corresponding signals to a motor driver 716.

The controller 704 is configured to communicate with a variable gain amplifier control 718, which may allow for control of a gain factor of a variable gain amplifier 726 that is part of a radiofrequency (RF) signal processing circuit 720. The RF signal processing circuit 720 includes a buffer 722 coupled to two of the slip rings in slip rings 710 to receive RF signals from the coupled intravascular device. From the buffer 722, the received RF signals are filtered by a filter 724 before passing through the variable gain amplifier 726, the gain of which is controlled by the controller 704 through the variable gain amplifier control 718. Some embodiments of the rotational handpiece 700 may include a plurality of filters like filter 724. For example, some embodiments of the handpiece 700 include filters having center frequencies of 10, 20, and 40 MHz. Other embodiments may further include filters having center frequencies of 60 and 80 MHz for harmonic imaging. After passing through the RF signal processing circuit 720 the processed RF signal is transmitted over the communication link 406 to the BIB 800.

The controller 704 is coupled to an interface logic 728 to that provides for logical interfacing with the BIB 800 over the communication link 406. As depicted, the interface logic 728 receives messages from and transmits messages to the BIB 800 over a serial port. In the depicted embodiment, the serial port is an RS-232 type serial port with a data rate of 115 kHz, but other serial communication standards, ports, and data rates may be used in other embodiments.

A number of other features of the rotational handpiece 700 are depicted in FIG. 7. Many of these features may be readily understood by one of skill in the art from FIG. 7 alone or in connection with the other figures of the present disclosure. Further detailed discussion of such features is omitted so as to maintain the clarity of this disclosure.

Referring now to FIG. 8, a more detailed illustration of the bedside interface box 800 introduced in FIG. 4 is illustrated therein. As depicted in FIG. 8, the BIB 800 includes the physiology device connector 810, the solid-state imaging device connector 820, and the rotational imaging device connector 830. Each of these connectors 810, 820, and 830 includes a physical interface and electrostatic discharge protection, and each of the connectors includes a plurality of individual communication lines over which signals are transmitted and received and by which power and ground are supplied to intravascular devices when coupled to the connectors. The connectors 810, 820, and 830 may be hot pluggable in some embodiments.

The physiology device connector 810 is connected to a physiology device interface circuit 812 that provides an amount of signal processing or signal preprocessing to signals received from a coupled intravascular physiology device. As depicted, the physiology device interface circuit 812 includes a digital output coupled to a controller 840, and an analog output coupled to an analog-to-digital converter 814 that, in turn, is coupled to the controller 840. In some embodiments, only a digital output or an analog output is provided by the physiology device interface circuit 812.

The solid-state imaging device connector 820 is coupled to a solid-state imaging device interface circuit 822. Like the physiology interface device circuit 812, the solid-state imaging device interface circuit 822 is configured to provide signal processing to signals received from a coupled solid-state imaging device. In some embodiments, preprocessing is performed by the solid-state imaging device interface circuit 822, and thereafter the preprocessed signals are provide to the controller 840 for further processing. RF signals received through the solid-state imaging device connector 820 are transmitted to a buffer 824 that is included in an IVUS RF signal processing circuit 850. The IVUS RF signal processing circuit 850 further includes an additional buffer 834 that is configured to receive RF signals from a rotational imaging device coupled to the rotational imaging device interface connector 830.

Thus, the IVUS RF signal processing circuit 850 provides preprocessing for RF signals received on either the solid-state imaging device connector 820 or the rotational imaging device connector 830. The controller 840 is coupled to the IVUS RF signal processing circuit 850 by a device select line that allows for the selection of signals from buffer 824 or from buffer 834. The IVUS RF signal processing circuit 850 further includes a plurality of filters, such as bandpass filters 852 and 854 that are selected by the controller 840 through a filter select line depending on whether the controller 840 has selected to receive signals from buffer 824 or buffer 834. Due to differences in the RF signals received from a solid-state imaging device compared to a rotational imaging device, a filter 852 may be suitable for signals received on buffer to 824, while the filter 854 may be suitable for signals received on the buffer 834. The plurality of filters within the IVUS RF signal processing circuit 850 are coupled to a variable gain amplifier 856. Some embodiments of the BIB 800 may include more than two band-pass filters as depicted in FIG. 8. For example, an embodiment of BIB 800 includes filters having center frequencies of 10, 20, and 40 MHz. Other embodiments may include filters having center frequencies of 60 to 80 MHz for harmonic imaging. The variable gain amplifier 856 amplifies receive signals in accordance with a gain controller 858 that is coupled to the controller 840. Signals output from the variable gain amplifier 856 are transmitted to an analog-to-digital converter 860 before being transmitted to the controller 840 for further processing.

As depicted in FIG. 8, signal processing may occur at various points within the BIB 800. In some embodiments, some or all of the signal processing may be performed by the controller 840. Controller 840 is one or more processors in communication with a memory 842. In some embodiments, one or more of the one or more processors is provided by a programmable processor, such as an FPGA or PLD as discussed above in connection with controller 704 of FIG. 7. Multiple configurations for an FPGA-based controller 840 may be stored in the memory 842 and implemented depending on the intravascular device or devices coupled to connectors 810, 820, and or 830. For example, in a situation in which no intravascular device is coupled to the physiology device connector 810, a configuration stored in 842 may be implemented by 840 such that no processing circuitry is implemented in controller 840 for physiological data, such as would be received on the physiology interface device connector 810.

As another example, a first configuration stored in memory 842 may be implemented in the controller 840 upon connection of a solid-state imaging device to the solid-state imaging device connector 820. This first configuration selects buffer 824 using the device select line, and selects filter 852 using the filter select line, and adjusts the gain control to tailor the performance of the variable gain amplifier 856 to better process solid-state IVUS imaging data. When a rotational intravascular device is connected to the rotational intravascular device connector 830, a second configuration stored in memory 842 may be implemented in the controller 840 such that the buffer 834 is selected along with the filter 854, rather than the buffer 824 and the filter 852. In this way, the FPGA-based controller 840 may be reconfigured to enable and/or optimize performance of the BIB 800 according to the intravascular devices connected thereto. BIB 800 further includes an intravascular device type and wire detection mechanism.

The BIB 800 further includes a cable interface 870 configured to receive power and ground for the BIB 800 as well as connected devices, such as the rotational handpiece 700. The cable interface 870 includes an isolated DC-to-DC conversion circuit 872 and a BIB/PIM power generation circuit 874 that provides power conditioned for the BIB 800 and connected devices. The cable interface 870 is further configured to transmit and receive data and control signals with an external device, such as the console 116 depicted in FIGS. 1 and 2. In some embodiments, like that depicted in FIG. 8, the cable interface 870 includes a fiber optic transceiver, such as the small-form-factor pluggable transceiver 876. By transmitting and receiving data over a fiber optic link, the BIB 800 may transmit data and receive full-duplex data that is less degraded due to the electromagnetic ambience in the area in which the BIB 800 is being used and/or due to extended cable lengths. Such an area may be the catheter lab 102 of FIG. 1.

Certain features of the BIB 800 are depicted in FIG. 8, but not described in detail herein. Such features may be apparent to those of skill in the art, and may be understood without verbose description. For the sake of clarity in this discussion, the description of such details is omitted.

Referring now to FIG. 9, shown therein is an intravascular device system 900 that is similar in many respects to the intravascular device system 200 of FIG. 2. For instance, the intravascular device system 900 includes a console 116 in communication with a display 106. FIG. 9 also illustrates an intravascular device interface 902 that is similar in many respects to the intravascular device interfaces depicted in FIGS. 1, 2, 3, and 4. In some instances the intravascular device interface 902 is connected to a sled 204 and is coupled to the console 116 and or the display 106 by a communication cable 220.

The intravascular device interface 902 includes an intravascular device connector 904 that is configured to receive either a non-rotational imaging device 906 or a rotational imaging device 908 therein. Both the non-rotational imaging device 906 and the rotational imaging device may feature a four-wire data interface. For example, in some implementations the rotational imaging device utilizes circuit architectures similar to those described in U.S. Provisional Patent Application No. 61/646,062, filed May 11, 2012 and titled “CIRCUIT ARCHITECTURES AND ELECTRICAL INTERFACES FOR ROTATIONAL INTRAVASCULAR ULTRASOUND (IVUS) DEVICES,” which is hereby incorporated by reference in its entirety. Further, in some implementations the rotational imaging device utilizes circuit architectures similar to those described in U.S. Provisional Patent Application No. 61/746,804, filed Dec. 28, 2012 and titled “INTRAVASCULAR ULTRASOUND IMAGING APPARATUS, INTERFACE ARCHITECTURE, AND METHOD OF MANUFACTURING,” which is hereby incorporated by reference in its entirety.

In other instances, the intravascular device connector 904 includes an adjustable connector that can adjust the number of electrical contacts (and associated processing) to match the characteristics of the received intravascular imaging device. In yet other instances, the intravascular device connector 904 includes a universal connector arrangement that is configured for use with intravascular imaging devices having different numbers of electrical contacts. Such a universal connector arrangement may require up to eight pins or more. This is particularly true for older generation intravascular devices. But embodiments of the intravascular device connector 904 may also include fewer pins. For example, if a particular rotational intravascular device uses four pins and a particular non-rotational intravascular device uses five pins, a five-pinned embodiment of the intravascular device connector 904 could be configured to provide an interface to both devices. Many different intravascular devices may be supported by an intravascular device connector 904 having five or six pins. By being able to receive both the non-rotational imaging devices and rotational imaging devices, the intravascular device interface 902 may obviate the need of buying and storing multiple intravascular device interfaces. Further detail of the intravascular device interface 902 is provided in FIG. 10.

FIG. 10 depicts the intravascular device interface 902 of FIG. 9 as coupled to an intravascular device 1002 through the intravascular device connector 904. The intravascular device 1002 may be either a rotational imaging device such as rotational imaging device 908 or a non-rotational imaging device, such as non-rotational imaging device 906. As depicted, the intravascular device connector 904 is coupled to a plurality of slip rings 1004 and to a motor 1006. When the intravascular device 1002 is coupled to the intravascular device interface 902 through the connector 904, a controller 1010 detects an identifying characteristic of the intravascular device that indicates whether the intravascular device is a rotational imaging device or a non-rotational imaging device.

When the intravascular device 1002 is a rotational imaging device, the controller 1010 communicates with a motor control 1012 to enable the motor 1006. As the motor 1006 is coupled to the intravascular device 1002, the motor is enabled to provide rotation to at least a portion of the intravascular device 1002 (e.g., a drive cable coupled to an ultrasound transducer). However, when the controller 1010 detects an identifying characteristic indicating that the intravascular device 1002 is a non-rotating intravascular device, the controller 1010 communicates with a rotation preventer 1014, which provides or facilitates a rotation prevention mechanism within the intravascular device interface 902. In some embodiments, the controller 1010 communicates with a motor control 1012 to prevent rotation of the coupled intravascular device 1002, thus providing a rotation prevention mechanism. In other embodiments, the rotation preventer 1014 is a mechanical structure such as a block or key in communication with the connector 904, which physically prevents the intravascular device 1002 from rotating. Thus, even in the event of a malfunction in the controller 1010, the mechanical structure of the rotation preventer 1014 ensures that the non-rotational intravascular device 1002 does not rotate. When the controller 1010 determines the identifying characteristic, it may perform a look-up into memory 1016 to determine the significance or meaning of the identifying characteristic or to identify an identifying characteristic value stored in an EEPROM or other memory.

As depicted, the slip rings 1004 are coupled to a high voltage supply 1018 to receive a supply voltage and a ground. In some implementations, the slip rings 1004 further provide a four-wire interface for communication between the coupled intravascular device 1002 and the intravascular device interface 902. As depicted, this four-wire interface includes two wires connected to the controller 1010 and two wires connected to a protection circuit 1020. From the protection circuit 1020, a signal path to a signal processing circuit 1030 further includes an amplifier 1022 a plurality of bandpass filters 1024, and analog to digital converter 1026.

As depicted, the intravascular device interface 902 includes a separate controller 1010 and signal processing circuit 1030. In some embodiments, the controller 1010 and the signal processing circuit 1030 are provided by one or more processors. As depicted, each of the controller 1010 and the signal processing circuit 1030 is a programmable processor, such as an FPGA. Further, in some such embodiments, a single FPGA provides both the controller 1010 and the signal processing circuit 1030. With an FPGA-based controller 1010 and an FPGA-based signal processing circuit 1030, certain aspects of the intravascular device interface 902 can be configured to enable and/or optimize performance for either a rotational intravascular device or a non-rotational intravascular device.

For example, the FPGA-based controller 1010 can implement a first configuration when the intravascular device 1002 is identified as a rotational intravascular device. This first configuration may include a specific gain setting on the amplifier 1022, a specific selection among the bandpass filters 1024, and an encoder 1007 that is coupled to the motor 1006 may be enabled by the configuration to provide rotational information to the controller 1010. In some embodiments, the first configuration may include a selection from band-pass filters 1024 having center frequencies of 10, 20, 40, 60, and 80 MHz. Similarly, a second configuration may be implemented by the controller 1010 and the signal processing circuit 1030 when the intravascular device 1002 is identified as a non-rotational intravascular device. The first and second configurations may also differ in a sampling rate used by the analog-to-digital converter 1026. For example, in the first configuration may include a sampling frequency of 160 MHz, while the second configuration includes a sampling frequency of 80 MHz.

The intravascular device interface 902 includes an output 1032 which transmits information to an external device. The intravascular device 902 further includes an isolation and power circuit 1034 that is configured to receive power and or signals from an external device.

Referring now to FIG. 11, therein is shown a flowchart of a method 1100 for using an intravascular imaging device to gather imaging data from within a patient. During the method 1100 the intravascular imaging device is coupled to an intravascular device interface, such as the intravascular device 902 as depicted in FIGS. 9 and 10. The method 1100 includes a plurality of steps as depicted, but embodiments thereof may include further steps before, in between, and/or after the depicted steps. As depicted, the method 1100 begins in a step 1102 in which the intravascular device interface receives an intravascular imaging device in a first connector. In step 1104, the intravascular device interface determines whether the intravascular imaging device received in the first connector is a rotational intravascular imaging device or a non-rotational intravascular imaging device. Depending on whether the intravascular device interface determines that the intravascular imaging device is a rotational or non-rotational intravascular imaging device, the intravascular device interface configures operational features, in step 1106.

Embodiments of the method 1100 may further include using a type-identification circuit to determine whether the intravascular imaging device is a rotational or non-rotational type intravascular imaging device. Configuring operational features on the intravascular device interface is performed by implementing one of a plurality of configurations on an FPGA-based controller. The plurality of configurations may be stored in a memory in communication with the FPGA-based controller. Additionally, configuring the operational features on the intravascular device interface may include implementing one of a plurality of configurations stored in memory for an FPGA-based signal processing circuit.

Referring now to FIG. 12, shown therein is a flowchart of a method 1200 for collecting multiple types of data from multiple intravascular device types within a patient. As depicted in FIG. 12, the method 1200 includes a plurality of steps. Many embodiments of the method 1200 further include additional steps before, in between, and/or after the enumerated steps without departing from the scope of this disclosure. As depicted, method 1200 begins in step 1202 in which an intravascular imaging device is coupled to intravascular device interface, such as the intravascular device 202 of FIG. 2, intravascular device 300 of FIG. 3, and/or the BIB 800 as depicted in FIGS. 4 and 8. In step 1204, an intravascular physiology sensing device is also coupled to the intravascular device interface. Both the intravascular imaging device and the intravascular physiology-sensing device may be used concurrently within a patient. In step 1206, imaging data is collected using the intravascular imaging device; while in step 1208 the physiological data is collected using the intravascular physiology-sensing device. Using one or more processors provided by the intravascular device interface the imaging data and the physiological data are processed, in step 1210.

In some embodiments of the method 1200, the intravascular device interface includes a plurality of intravascular device connectors that may be used to receive different types of intravascular devices. In some embodiments, the intravascular device interface includes at least one intravascular imaging device connector and at least one intravascular physiology-sensing device connector. In some related embodiments, the intravascular device interface includes a non-rotational imaging device connector as well as a rotational imaging device connector. Some embodiments of the method 1200 further include transmitting the processed imaging data and physiological data to a console over a fiber optic link. In other embodiments, the method 1200 further includes transmitting the processed imaging data and physiological data to a display. Additionally, the intravascular device interface if make be configured with a first configuration based on a type of the intravascular imaging device.

The examples provided above are exemplary only and are not intended to be limiting. The embodiments provide a universal intravascular device interface, or a universal PIM, for use in intravascular device systems. One skilled in the art may readily devise other systems consistent with the disclosed embodiments which are intended to be within the scope of this disclosure. As such, the application is limited only by the following claims.

What is claimed is:

1. An intravascular device interface, comprising:
a housing containing one or more processors in communication with a memory;
a first connector secured to the housing and configured to interface with a proximal connector of an imaging intravascular device, the imaging intravascular device being sized and shaped for insertion into human vasculature;
a second connector secured to the housing and configured to interface with a proximal connector of a physiology intravascular device, the physiology intravascular device being sized and shape for insertion into human vasculature, and wherein the one or more processors process data received from the first connector or the second connector for transmission over a device output.

2. The device of claim 1, wherein the second connector is configured to support a plurality of different physiology intravascular device types.

3. The device of claim 2, wherein the second connector is configured to support a pressure-sensing physiology intravascular device.

4. The device of claim 2, wherein the second connector is configured to support a flow-sensing physiology intravascular device.

5. The device of claim 2, wherein the second connector is configured to support a pressure-sensing and flow-sensing physiology intravascular device.

6. The device of claim 1, wherein the first connector is configured to interface with a rotational imaging catheter.

7. The device of claim 6, wherein the rotational imaging catheter is a rotational intravascular ultrasound (IVUS) catheter.

8. The device of claim 6, wherein the rotational imaging catheter is an optical coherence tomography (OCT) catheter.

9. The device of claim 6, further comprising a third connector configured to interface with a proximal connector of another imaging intravascular device.

10. The device of claim 9, wherein the first connector and the third connector are configured to receive different types of imaging intravascular devices.

11. The device of claim 9, wherein:
the first connector is configured to interface with a rotational imaging catheter; and
the third connector is configured to interface with a solid-state imaging catheter.

12. The device of claim 6, wherein the first connector is in communication with a rotary mechanism, the rotary mechanism configured to impart rotation to at least a portion of a rotational imaging intravascular device.

13. The device of claim 1, further comprising an intravascular ultrasound (IVUS) radiofrequency (RF) signal processing circuit coupled to the first connector.

14. The device of claim 13, wherein the IVUS RF signal processing circuit comprises:
a plurality of buffers in communication with the first connector and the third connector to receive RF input signals from the first connector or the third connector;
a plurality of filters coupled to the plurality of buffers to filter a signal received from one or the plurality of buffers; and
a variable gain amplifier coupled to the plurality of filters and to a gain controller to amplify a signal received from one of the plurality of filters.

15. The device of claim 14, wherein the IVUS RF signal processing circuit further comprises:
a catheter select line coupled to the plurality of buffers to select an output from one of the plurality of buffers and thereby to select an output from one of the first connector and the third connector; and
a filter select line coupled to the plurality of filters to select one of the plurality of filters for use by the IVUS RF signal processing circuit, wherein the catheter select line, the filter select line, and the gain controller are coupled to the one or more processors.

16. The device of claim 1, wherein the device output includes a fiber optic transceiver in communication with the one or more processors.

17. An intravascular device system comprising:
an intravascular device interface that has a first connector configured to interface with a proximal connector of an imaging intravascular device and a second connector configured to interface with a proximal connector of a physiology intravascular device;
a rotational handpiece coupled to the intravascular device interface;
a sled supporting the intravascular device interface so that the intravascular device interface is controllably moveable along an axis; and
a plurality of user controls in communication with the rotational handpiece.

18. The intravascular device system of claim 17, wherein the intravascular device interface further comprises:
a housing containing one or more processors in communication with a memory, and wherein the first connector and the second connector are secured to the housing and are coupled to the one or more processors such that the one or more processors processes data received from the second connector or the first connector for transmission over a system output.

19. The intravascular device system of claim 17, wherein the one or more processors are configured to simultaneously process data received from a physiological intravascular device and data received from the imaging intravascular device.

20. The intravascular device system of claim 17, wherein the intravascular device interface comprises third connector, and wherein the first connector provides an interface for a rotational intravascular imaging device and the third connector provides an interface for a non-rotational intravascular imaging device.

21. A method for using an intravascular imaging device to gather imaging data from within a patient, the intravascular imaging device being coupled to an intravascular device interface, the method comprising:
receiving an intravascular imaging device in a first connector provided on the intravascular device interface;
determining whether the intravascular imaging device received in the first connector is a rotational intravascular imaging device or a non-rotational intravascular imaging device; and
configuring operational features on the intravascular device interface depending on the determination of whether the intravascular imaging device is a rotational intravascular imaging device or a non-rotational intravascular imaging device.

22. The method of claim 21, wherein determining whether the intravascular imaging device received in the first connector is a rotational intravascular imaging device or a non-rotational intravascular imaging device comprises using a type identification circuit.

23. The method of claim 21, wherein configuring operational features on the intravascular device interface comprises implementing one of a plurality of configurations on an FPGA-based controller.

24. The method of claim 21, wherein configuring operational features on the intravascular device interface comprises implementing one or a plurality of configurations on an FPGA-based signal processing circuit.

25. The method of claim 21, wherein the operational features comprise:
a motor enablement status;
a central frequency of a bandpass filter section; and
a sampling rate of an analog-to-digital converter.

26. The method of claim 21, further comprising:
receiving imaging data from the intravascular imaging device;
processing the received image data with a signal processing circuit in a first configuration when the intravascular imaging device is a rotational intravascular imaging device; and
processing the received imaging data with a signal processing circuit in a second configuration when the intravascular imaging device is a non-rotational intravascular imaging device.

27. The method of claim 21, wherein the first connector provides a four-wire data interface.
